(19) United States
(12) Patent Application Publication
(10) Pub. No.: US 2026/0321431 A1
(43) Pub. Date: Oct. 15, 2026

(54) System and method for identifying high-risk individuals for prioritized communication

(71) Applicants: Ajay Dharod, Winston-Salem, NC (US); David P. Miller, JR., Winston-Salem, NC (US)

(72) Inventors: Ajay Dharod, Winston-Salem, NC (US); David P. Miller, JR., Winston-Salem, NC (US)

(21) Appl. No.: 19/462,948

(22) Filed: Jan. 28, 2026

Related U.S. Application Data
(60) Provisional application No. 63/552,080, filed on Feb. 9, 2024.

(51) Int. Cl. G16H 50/30; G16H 10/20; G16H 20/70

(52) U.S. Cl. CPC G16H 50/30; G16H 10/20; G16H 20/70

(57) ABSTRACT

System and method for identifying high-risk individuals for prioritized communication. The system preferably sends questions and receives answers to determine a user's risk and annotates when a user is high risk to prioritize communication with them.

### CROSS-REFERENCE TO RELATED APPLICATIONS

This application claims priority to pending application 63/552,080 filed on Feb. 9, 2024.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### REFERENCE TO A “SEQUENCE LISTING,” A TABLE, OR A COMPUTER PROGRAM LISTING APPENDIX SUBMITTED ON COMPACT DISC AND AN INCORPORATION-BY-REFERENCE

### OF THE MATERIAL ON THE COMPACT DISC

Not Applicable

### STATEMENT REGARDING PRIOR DISCLOSURES BY AN INVENTOR OR JOINT INVENTOR

Not Applicable

### BACKGROUND OF THE INVENTION

An important role of medical screenings is to identify the urgency with which an individual should be seen. In an emergency room, patients are often assigned one of five levels on the Emergency Severity Index (ESI). These levels are Level 1 (most urgent), level 2 (urgent), level 3 (moderate urgency) level 4 (low urgency) and level 5 (least urgent). By assigning an individual an appropriate urgency level, scare resources can be allocated where they are most required and will do the most good.

There can be disagreement between patients and medical staff on the level of urgency, in both directions. For example, a patient may underestimate the level of urgency and believe they are suffering significant heartburn when medical staff may be concerned about potential heart attack. On the other hand, a patient may believe they have very urgent need for attention believing they have a broken bone when medical staff may believe the patient is suffering from a sprain which is stable and unlikely to get worse in the short term.

Helping patients understand the appropriate urgency of seeking care can help patients advocate for themselves. Identifying the level of urgency can also help medical care providers perform follow-up on an appropriate basis and prioritize communication with those patients who require services most urgently.

### DESCRIPTION OF RELATED ART INCLUDING INFORMATION DISCLOSED UNDER 37 CFR 1.97 AND 37 CFR 1.98

Not Applicable

### BRIEF SUMMARY OF THE INVENTION

System and method for identifying high-risk individuals for prioritized communication. The system preferably sends questions and receives answers to determine a user's risk and annotates when a user is high risk to prioritize communication with them.

### BRIEF DESCRIPTION OF THE SEVERAL VIEWS OF THE DRAWING

FIG. 1 shows a system of the present invention.

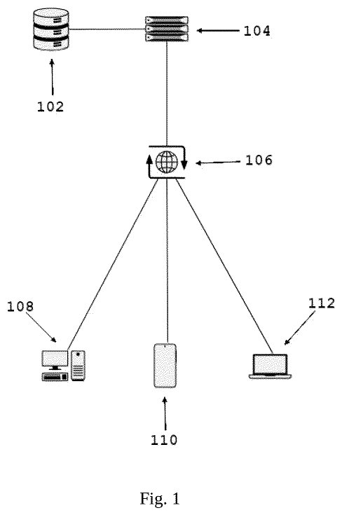

FIG. 2 shows an authentication process of the present invention.

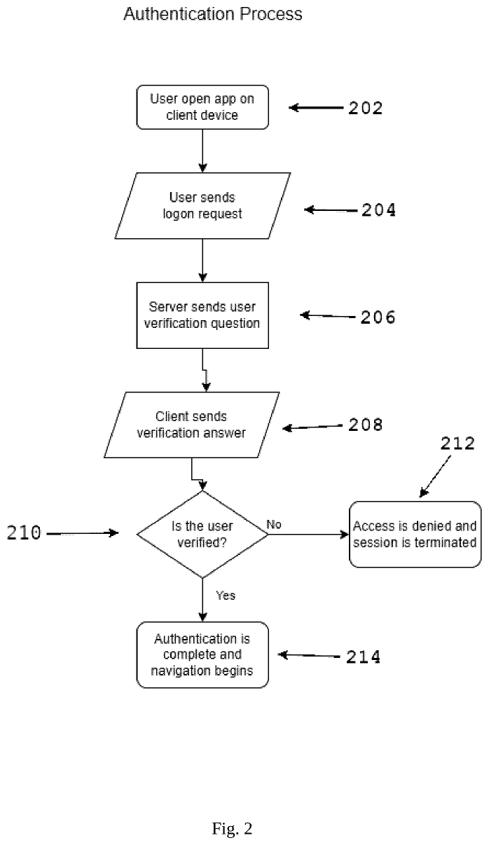

FIG. 3 shows a navigation process of the present invention.

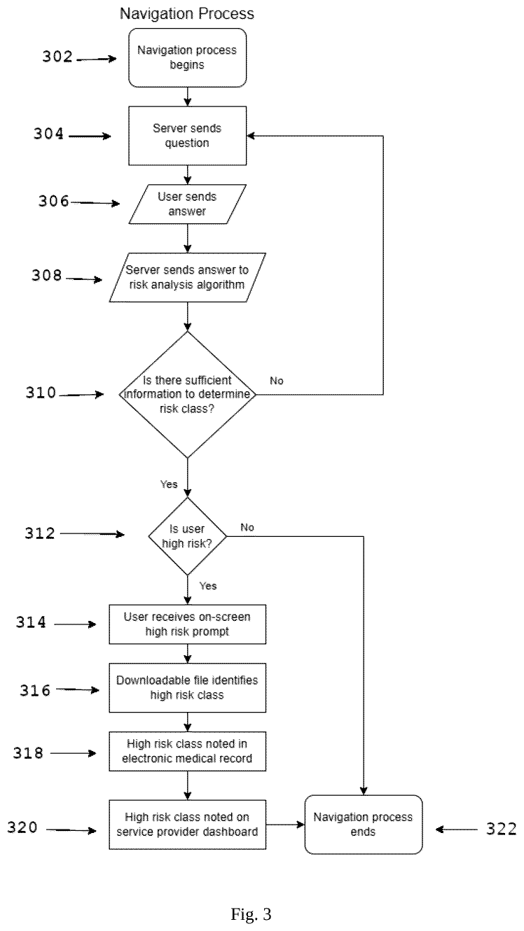

### DETAILED DESCRIPTION OF THE INVENTION

The system of the present invention preferably comprises computer readable media 102 configured with instructions to cause the server 104 to carry out proscribed functions. The server 104 is preferably connected to the internet 106. Client devices such as computers 108, mobile telephones 110, and laptops 112 are also preferably connected to the internet 106. A user of a client device 108, 110, or 112 may access a web page of a web application, run a compiled or other application, or otherwise send a command to the server to initiate an interactive process. The server 104 may be a single server or a collection of servers configured to carry out the desired process.

In a preferred embodiment, the process begins by authenticating the user to determine whether the user is who they assert to be. This begins with a user opening an application or accessing a web page 202. The user then sends a login request 204. This may take the form or entering a name, email address, telephone number, or accessing a specifically provided link. The server then preferably sends a follow-up question 206 to verify whether the user knows something only the authentic user would know. The client preferably answers this question sending a response back to the server 208. The server then checks to see whether this answer matches the answer known at the server 210. If the answer does not match, the user is not verified and access is terminated 212. If the answer matches, the user is verified and the user may then progress to the navigation process 214.

The server 104 reads instructions from the computer readable media 102 to select a question. The question is then sent 304 to a client device 108, 110, or 112. The client device then displays to a user, on an electronic graphical user interface, the question. The user enters an answer to the question which is then sent back to the server 306. The server then sends the question and answer to a rules engine or algorithm 308. The answers to certain questions may, by themselves, place a user in a specific risk class. For example, asking a user's age and receiving an answer of fifteen may place a person at low risk category for arthritis. On the opposite end of the spectrum, asking whether someone has a BRCA1 or BRCA2 mutation and receiving the answer of yes may place someone in a high-risk category for breast cancer. In other situations, a series of questions and answers may be required to categorize a user's risk. On such algorithm is breast cancer risk assessment tool known as the Gail Model. In the best known method at the time, a sufficient number of questions are asked, and answers received, such that the algorithm can determine a risk category and the question and answer process terminates immediately when the risk category has been

### Determined 310.

High-risk users are treated uniquely because of their risk. If a user is determined to be high-risk, the user promptly informed they are high-risk in the electronic graphical user interface 314. The user is also preferably offered the opportunity to download details of their questions, answers, and results including a notation that the user is high-risk 316. The high-risk classification is noted in the user's electronic health record 318. The high risk user(s) are also preferably noted on a service provider dashboard to facilitate prioritized communication 320. Once all required notations are made, the navigation process ends 322.

In a preferred embodiment, the present invention comprises a plurality of client computing apparatuses. Exemplary client computing apparatuses include mobile telephones, tablets, computers, laptops, kiosks, and telephones. The client computing apparatuses are configured to display information and accept user input. The present invention preferably also comprises one or more server computing apparatuses connected to one or more computer readable media. The computer readable medium preferably contains computer readable instructions which, when executed by the server computing apparatuses, carry out processes and methods of the invention. The plurality of server computing apparatuses is preferably connected to a communication medium. This communication medium may be an ethernet network, LAN, WAN, Wi-Fi, IDSN, T-1, DOCSIS, modem, or any other means known in the art by which computing devices may communication between and among each other. In a preferred embodiment, the client computing apparatuses are communicatively connected, directly or indirectly, to the plurality of server computing apparatuses such that the plurality of server computing apparatuses may send information to the plurality of client computing apparatuses and the plurality of client computing apparatuses may send information to the plurality of server computing apparatuses.

In a preferred embodiment, the instructions stored on the computer readable media instruct the server computing apparatuses to carry out steps comprising one or more of the following:
accept input identifying service consumers,
store information regarding service consumers,
schedule notices to be sent to service consumers via client computing apparatuses,
send notices to client computing apparatuses,
accept verification information from service consumers via client computing apparatuses,
present education information to service consumers via client computing apparatuses,
send requests for input to service consumers via client computing apparatuses,
accept input from service consumers via client computing apparatuses,
save input from service consumers,
read a plurality of rule sets,
feed input from service consumers into the plurality of rule sets,
send results of the rule sets to service consumers via client computing apparatuses, and
send results of the rule sets, and/or input from service consumers, to service providers.

By carrying out the above steps without the synchronous involvement of individuals opposite the service consumers, the number of service consumers who may be served scales much greater than when the process is conducted manually.

For purposes of this disclosure, service provider refers any individual or entity who provides or coordinates screening services. Service providers include, but are not limited to, medical service providers (e.g. doctors, nurses, etc.), health systems, accountable care organizations, insurers, retail health clinics, employers who provide health care, and other similar individuals or entities who may assist in scheduling, ordering, educating individuals regarding medical care, or providing health care. For purposes of this disclosure, service consumer refers to an individual who may undergo a preventative screening process or an individual who may be assisting an individual who may undergo a preventative screening process in filling out forms, providing advice, or providing other assistance.

For purposes of this disclosure, a preventative screening includes, but is not limited to, questionnaires, imaging, laboratory tests, cultures, and other means for determining the presence or absence of a looked-for attribute. In a preferred embodiment, the attribute is a health condition, such as a cancerous state. Other attributes such as presence of a communicable health condition such as tuberculosis, or a non-communicable medical condition such as diabetes, are also within the definition of an attribute. Lastly, attribute also includes other mental, physical, and non-physical states such as addictions.

In a preferred embodiment, the system and process are used to educate and screen service consumers for preventative screenings. These preventative screenings may include, but are not limited to, cholesterol measurements, fecal occult blood tests, pap tests, prostate specific antigen evaluations, mammographies, colonoscopies, diabetes or prediabetes screening, or low-dose computed tomography scans. Other processes, such as routine physical examinations, are within the scope of the present invention though the present invention is described in greater detail as it relates to certain preventative screening processes.

In a preferred embodiment of the present invention, the method and apparatus are used to identify service consumers who may need, or benefit from, a preventative screening, notify the service consumers of their potential need/eligibility, verify the identity of service consumers, verify the need/eligibility for one or more preventative screening processes, leverage behavioral change principles to educate and motivate the service consumers to participate in a preventative screening, offer personalized risk/benefit prediction modeling, allow service consumers to self-select one or more from a list of available preventative screening processes, and automatically transmit information to a service provider. Personalized risk/benefit modeling relates to the condition being screened for, for harms/benefits from undergoing one or more preventative screening processes, and/or for electing not to undergo one or more preventative screening processes. In certain embodiments, rule sets, algorithms, machine learning, surveys, and other means may be used to identify service consumers who may be well-suited to participate in an automated pre-screening or patient navigation process, be likely candidates for a preventative screening, and/or be willing to participate in an automated process.

In one embodiment, a service provider enters information into the apparatus, or initiates a process, via a semi-manual process for individual/patient identification. In this embodiment, data accuracy and data missingness may be manually assessed by a database analyst on behalf of the service provider, or by a rule set. Subsequently, the service provider transfers several pertinent data elements to the apparatus of the present invention 104 informing the apparatus'subsequent embodiments/functions.

In an alternative embodiment of identification and interaction with the apparatus of the present invention, the apparatus is directly connected to a service provider's database. This connection may be temporary for a short or long time, or may be persistent. These service provider databases may include electronic health record information, a data warehouse, or other databases. In this embodiment, structured, detailed inclusion/exclusion criteria are applied to the customer database via a database query with automatic transmission of returned data-elements to the system of the present invention informing the system's subsequent embodiments/functions. In this embodiment, data accuracy and data missingness are assessed in a semi-automated fashion using algorithms.

In a preferred embodiment, selected service consumers receive one or more notifications of the potential need/benefit for a preventative screening through one or more motivational/invitational messages sent to the service consumer(s). In a preferred embodiment, the plurality of motivational messages are sent electronically to client devices, or accounts, of the service consumers. In a preferred embodiment, these motivational/invitation messages may be scheduled 106 to be delivered at a desired time or time range. In an alternative embodiment, the motivational/invitational message is sent in physical form and includes a mechanism, such as a QR code or hyperlink, to access the automated process. The content of the message may vary based on geographic, demographic, or individual patient-level variables. If no additional messages are scheduled to be sent, the motivational/invitational message portion of the process is complete. Alternatively, electronic message content may be defined by the service provider. In another embodiment, reminder text messages may be sent to service consumers at fixed or relative intervals with a final reminder message sent a defined time prior to an upcoming visit with a service provider. In these embodiments, information provided by the service provider may be embedded within the message content to increase the service consumer's trust in the motivational/invitational message to participate in the automated process of the present invention. In another embodiment, service consumers identified by the service provider may receive a notification of the need/benefit for a preventative screening through a message to the service consumer in a portal operated by/for the service provider.

In a preferred embodiment, the identity of the service consumer is verified at the outset of the automated process. The service consumer preferably responds to a message and accepts the invitation to participate in the automated process. The server device then sends a plurality of identity verification questions to the service consumer's client device. The service consumer then enters answers to the plurality of identity verification questions. If the answers to the plurality of identity verification questions are correct, the service consumer is then directed to continue through the automated process. If the answers to the plurality of identity verification questions is/are incorrect, the service consumer's progress through the automated process is terminated.

After the identity of the service consumer is verified, the service consumer then preferably continues through the automated process. In one embodiment of the present invention, the need/benefit/eligibility for a service consumer to undergo a preventative screening need is verified/determined through a series of questions, specific to each preventative screening (i.e. colorectal cancer, lung cancer, prostate cancer, etc.), which are sent to the service consumer and the service consumer answers. In this embodiment, each preventative screening employs a different set of questions tailored to the particular preventative screening processes and the system applies a predefined rule set which defines condition-specific criteria for preventative screening eligibility. This rule set may be customized by the service provider, such as medical service provider or an insurance provider, based on the services they provide, provider networks, or coverage criteria. If the rule set indicates the service consumer is not eligible for a preventative screening process, the automated process ends. If the rule set indicates the service consumer is eligible for a preventative screening process, the automated process continues. If the rule set indicates the service consumer is not eligible for a preventative screening process, the automated process preferably ends.

Barriers and facilitators are factors which respectively reduce or increase the likelihood of a patient undergoing a preventative screening process. Exemplary barriers include unavailability during regular hours (such as working a night shift), transportation challenges (not having a car and needing to access services at a location not serviced by public transportation), and financial (not being able to afford a particular preventative screening process). Exemplary facilitators include offering services during extended hours, offering services at various locations, offering free or subsidized transportation, and helping service consumers avail themselves of insurance, Medicare, Medicaid, financing, and low-cost clinics.

In a preferred embodiment, the service consumer is presented with one or more questions about what factors (which may be barriers and facilitators) are important to the service consumer when making preventative screening decisions. Exemplary factors include false positive rate of the preventative screening, false negative rate of the preventative screening, cost of the preventative screening, follow-up interval if the preventative screening is selected, etc. The service consumer preferably answers the presented questions. The service consumer is then presented with one or more preventative screening options using decision making factors. A few examples of presenting a preventative screening options using decision making factors are: 1) “You said cost is very important to you. A genetic test is generally not covered by insurance and costs, on average, $2,000 dollars, but a genetic test has one of the lowest false positive rates,” 2) “You said your time is very important to you. A fecal test can be done at home, on your own schedule,” 3) “You said minimizing testing is important to you. A negative colonoscopy test can minimize the need for follow-up testing for as long as a decade.” In a preferred embodiment of the invention, the system asks the service consumer whether the service consumer wishes to schedule a preventative screening 318. In certain embodiments, the system asks the service consumer about a range of preventative screening options which the service consumer may choose among. The service consumer then enters their preference of whether/which preventative screening he/she would like to schedule. In a preferred embodiment, this ends the automated process.

In one embodiment of the invention, multimedia information leveraging behavioral change principles is sent to the service consumer on a client device. In this embodiment, based on predetermined criteria used by the system of the present invention, each preventative screening interaction process displays a set of questions for the service consumer tailored to the criteria for screening eligibility for the specific condition screened for. Furthermore, within this embodiment, personalized risk prediction may be shown to the service consumer in a visual, audible, and/or video formats. In a preferred embodiment, the multimedia information is tailored to enhancing preventative screening facilitators and overcoming preventative screening barriers.

In a preferred embodiment, the service consumer information is stored within the system of the present invention. Results of the rule sets, or answers to specific questions (such as desire to schedule a specific preventative screening process) are provided to appropriate service providers. The ordering process may be manual or automated. In certain embodiments, orders for preventative screenings are automatically sent to a service provider. In this embodiment, no further steps are required in the ordering process. In another embodiments, the server sends the answers entered service consumers, or results of rule sets applied to the answers to a device for review by a service provider. The results, or answers, are preferably reviewed by the service providers. If the service consumer is eligible for the requested preventative screening process, the service provider then sends an order for the selected preventative screening processes to a service provider and the ordering process then ends. If the service consumer is not eligible for the requested preventative screening process, this ends the ordering process without an order being place.

In certain embodiments, methods of the present invention are carried out using accessibility technology. Accessibility technology includes, but is not limited to, screen readers which convert text to speech, speech-to-text converts which interpret voice responses by service consumers to text, Braille converters, and similar technologies.

For purposes of this application, including claims, plurality means one or more. All quantities are exemplary and other quantities are anticipated within the scope of the present invention unless otherwise indicated. Although described with particularity for high-risk users, the apparatus and method may also be practiced for users with other risk classifications. For example, although current methodologies are particularly useful when applied to two risk classes (high where a user's risk exceeds a defined particular threshold and other than high where a user's risk does not exceed a defined threshold), the system and apparatus could be equally applied with three or more risk classes. The particular threshold(s) is/are not critical the system and apparatus of the invention. Different thresholds may be used by different parties implementing the system and apparatus.

1. An automated method for identifying high-risk individuals for prioritized communication comprising:
A) sending a plurality of questions to an electronic graphical user interface for a user,
B) receiving answers to the plurality of questions,
C) send a plurality of answers into a risk analysis algorithm,
D) receiving a risk score from the risk analysis algorithm, and
E) identifying whether a user as high-risk based on the risk score calculated by the risk analysis algorithm.

2. The automated method of claim 1 further comprising:
A) sending a message to the electronic graphical user interface for the user that the user should promptly contact a service provider if the user is identified as high-risk.

3. The automated method of claim 1 further comprising:
A) saving in a file an indicator that the user should promptly contact a service provider if the user is identified as high-risk.

4. The automated method of claim 1 further comprising:
A) saving in an electronic health record system an indicator that the user should promptly contact a service provider if the user is identified as high-risk.

5. The automated method of claim 1 further comprising:
A) displaying information about the user in a relevant location on an electronic dashboard summarizing user responses if the user is identified as high-risk.

6. The automated method of claim 1 wherein:
A) the plurality of questions include questions about a user's clinical signs and symptoms.

7. The automated method of claim 1 wherein:
A) the plurality of questions include questions about a user's personal behavioral history.

8. The automated method of claim 1 wherein:
A) the plurality of questions include questions about a user's objective diagnostic investigation laboratory values and other medical test, procedures, and results.

9. The automated method of claim 1 wherein:
A) the plurality of questions include questions about a user's family medical history.

10. A computer-implemented system for identifying high-risk individuals for prioritized communication comprising:
A) computer-readable media in communication with a computer server apparatus connected to a telecommunication network wherein the computer-readable media contains instructions to:
I) send a plurality of questions to an electronic graphical user interface for a user,
II) receive answers to the plurality of questions,
III) send a plurality of answers into a risk analysis algorithm,
IV) receiving a risk score from the risk analysis algorithm, and
V) identify a user as high-risk based on the risk score calculated by the risk analysis algorithm.

11. The computer-implemented system of claim 10 wherein the computer-readable media further contains instructions to:
A) send a message to the electronic graphical user interface for the user that the user should promptly contact a service provider if the user is identified as high-risk.

12. The computer-implemented system of claim 10 wherein the computer-readable media further contains instructions to:
A) save in a file an indicator that the user should promptly contact a service provider if the user is identified as high-risk.

13. The computer-implemented system of claim 10 wherein the computer-readable media further contains instructions to:
A) save in an electronic health record system an indicator that the user should promptly contact a service provider if the user is identified as high-risk.

14. The computer-implemented system of claim 10 wherein the computer-readable media further contains instructions to:
A) display information about the user in a relevant location on an electronic dashboard summarizing user responses if the user is identified as high-risk.

15. The computer-implemented system of claim 10 wherein:
A) the plurality of questions include questions about a user's clinical signs and symptoms.

16. The computer-implemented system of claim 10 wherein:
A) the plurality of questions include questions about a user's personal behavioral history.

17. The computer-implemented system of claim 10 wherein:
A) the plurality of questions include questions about a user's objective diagnostic investigation laboratory values and other medical test, procedures, and results.

18. The computer-implemented system of claim 10 wherein:
A) the plurality of questions include questions about a user's family medical history.
